(19) United States
(12) Patent Application Publication
(10) Pub. No.: US 2020/0108188 A1
(43) Pub. Date: Apr. 9, 2020

(54) NEEDLE DRAWING ASSISTANCE APPARATUS

(71) Applicants: Joseph Bell, Apex, NC (US); Joan Bell, Apex, NC (US)

(72) Inventors: Joseph Bell, Apex, NC (US); Joan Bell, Apex, NC (US)

(21) Appl. No.: 16/299,224

(22) Filed: Mar. 12, 2019

Related U.S. Application Data
(60) Provisional application No. 62/740,485, filed on Oct. 3, 2018.

(51) Int. Cl. A61M 1/00; A61M 5/24

(52) U.S. Cl. CPC A61M 1/0078; A61M 5/148; A61M 5/2425

(57) ABSTRACT

A needle drawing assistance apparatus. The apparatus includes a first planar member defining a first end and a second end and a second planar member having a proximal end and a distal end. The second planar member is secured to the first end of the first planar member at the proximal end of the second planar member via a hinge. The hinge is configured to allow the needle assistance apparatus to be freely movable between an active position and an inactive position. The inactive position is defined where the first planar member rests parallel and in contact with the second planar member. The active position is defined where the first planar member defines an angle greater than zero degrees with the second planar member.

### CROSS REFERENCE TO RELATED APPLICATIONS

This application claims the benefit of U.S. Provisional Application No. 62/740,485 filed on Oct. 3, 2018. The above identified patent application is herein incorporated by reference in its entirety to provide continuity of disclosure.

### BACKGROUND OF THE INVENTION

The present invention relates to a needle drawing assistance apparatus. Specifically, a device designed to provide convenience for a user when extracting a liquid component from a storage element using a needle.

Many individuals are diagnosed with medical conditions which require them to perform an injection at certain times. Commonly, patients diagnosed with diabetes will need to make regular insulin injections to manage their blood sugar levels. It can be challenging, however, for these patients to extract insulin from the storage elements typically utilized to store insulin due to, among other reasons, interfacial tension that exists between a residual liquid component and the interior surface of a storage element, such as a vial.

Interfacial tension between the liquid and the storage apparatus may cause the patient to discard a storage element that still contains liquid components therein. This results in the waste of medical components, which may be expensive and not immediately available to replace. This is particularly problematic for patients dealing with serious medical conditions, such as diabetes due to the importance of maintaining proper blood sugar levels.

Therefore, there is a defined need amongst the known art for an apparatus that assists users when extracting remaining liquid components from a medical storage element such as a vial.

### SUMMARY OF THE INVENTION

In view of the foregoing disadvantages inherent in the known types of liquid extraction devices now present in the prior art, the present invention provides a liquid extraction device wherein the same can be utilized for providing convenience for the user when removing liquid components from a storage element.

The present system comprises a first planar member defining a first end and a second end and a second planar member having a proximal end and a distal end. The second planar member is secured to the first end of the first planar member at the proximal end of the second planar member via a hinge. The hinge is configured to allow the needle assistance apparatus to be freely movable between an active position and an inactive position. The inactive position is defined where the first planar member rests parallel and in contact with the second planar member. The active position is defined where the first planar member defines an angle greater than zero degrees with the second planar member.

A first clamp is disposed on a top surface of the first planar member adjacent to the second end of the first planar member. The first clamp is configured to removably secure a storage element, such as a vial, therein. A second clamp is disposed on the top surface of the first planar member between the first clamp and the first end of the first planar member. The second clamp is configured to removably secure a needle therein.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of an embodiment of the needle drawing assistance apparatus in an active position.

FIG. 2 shows a perspective view of an embodiment of the needle drawing assistance apparatus in an inactive position.

FIG. 3 shows a perspective view of an embodiment of the needle drawing assistance apparatus in use.

FIG. 4 shows a block diagram of an embodiment of the needle drawing assistance apparatus.

FIG. 5 shows a rear view of an embodiment of the needle drawing assistance apparatus.

### DETAILED DESCRIPTION OF THE INVENTION

Reference is made herein to the attached drawings. Like reference numerals are used throughout the drawings to depict like or similar elements of the needle drawing assistance apparatus. The figures are intended for representative purposes only and should not be considered to be limiting in any respect.

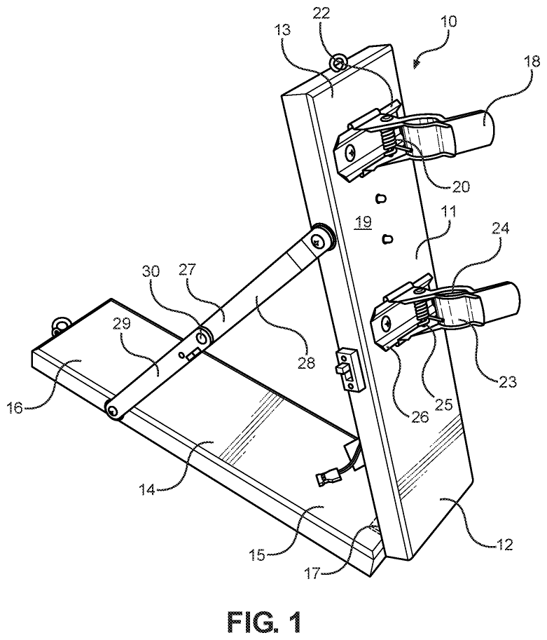

Referring now to FIG. 1, there is shown a perspective view of an embodiment of the needle drawing assistance apparatus in an active position. The needle drawing assistance apparatus 10 comprises a first planar member 11 defining a first end 12 disposed oppositely a second end 13. The first planar member 11 is made of a rigid material, such that elements disposed thereon are held in a non-movable position. The needle drawing assistance apparatus 10 further comprises a second planar member 14. The second planar member 14 has a proximal end 15 disposed oppositely a distal end 16. The second planar member 14 is made of a rigid material such that element disposed thereon are held in a non-movable position.

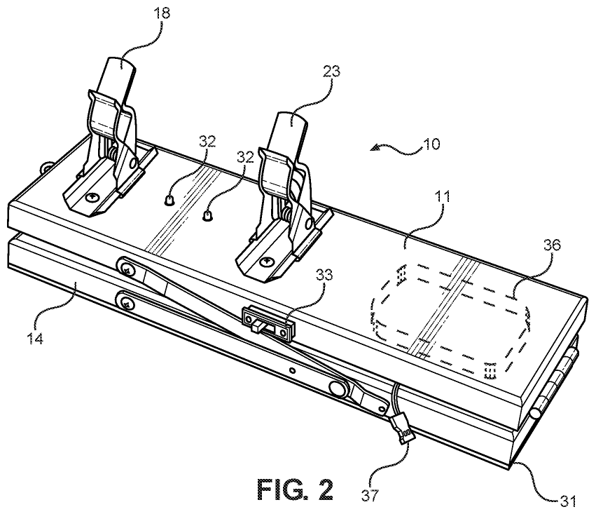

The second planar member 14 is secured at the proximal end 15 thereof to the first end 12 of the first planar member 11 via a hinge 17. The hinge 17 enables selective movement of the first planar member 11 relative to the second planar member 14 between an active position (shown in FIG. 1) and an inactive position (shown in FIG. 2). The active position is defined where the first planar member 11 defines an angle greater than zero degrees with the second planar member 14. Furthermore, the active position enables the user to utilize the force of gravity when removing liquid components from a storage element utilizing a needle.

A first clamp 18 is disposed on a top surface 19 of the first planar member 11. Specifically, the first clamp 18 is disposed between adjacent to the second end 13 of the first planar member 14. The first clamp 18 is configured to removably secure a storage element, such as a vial, therein. In the illustrated embodiment, the first clamp 18 is spring-biased in a closed position via a first clamp spring 20, such that a storage element is tensionably secured therein when the needle drawing assistance apparatus 10 in use. In the illustrated embodiment, the first clamp 18 is removably secured upon the first planar member 11 via engagement upon a first clamp track 22. Removable engagement with the first clamp track 22 allows for the first clamp 18 to be removed entirely from the first planar member 11, such as for storage or alignment.

A second clamp 23 is disposed on the top surface of 19 of the first planar member 11. Specifically, the second clamp 21 is disposed between the first clamp 18 and the first end 12 of the first planar member 11. The second clamp 23 is configured to removably secure a syringe of a needle therein. In the illustrated embodiment, the second clamp 23 comprises a padding 24 therein, such that additional security is provided to the needle when in use while preventing breakage of the needle when removably secured therein. Additionally, in the illustrated embodiment, the second clamp 23 is spring-biased in a closed position via a second clamp spring 25, such that the syringe of the needle is tensionably secured therein when the needle drawing assistance apparatus 10 is in use. In the illustrated embodiment, the second clamp 23 is removably secured upon the first planar member 11 via engagement upon a second clamp track 26. Removable engagement with the second clamp track 26 allows for the second clamp 23 to be removed entirely from the first planar member 11, such as for storage or for alignment with the first clamp 18.

In the illustrated embodiment, the needle drawing assistance device 10 comprises a brace 27. The brace 27 is configured to lock the position of the first planar member 11, such that the needle drawing assistance device 10 is secured in the active position. In the illustrated embodiment, the brace 27 is comprises a first member 28 rotatably secured to a side surface of the first planar member 11 and a second member 29 rotatably secured to a side surface of the second planar member 14. As shown, the first member 28 is secured to the second member by a rotatable joint 30.

Referring now to FIG. 2, there is shown a perspective view of an embodiment of the needle drawing assistance apparatus in an inactive position. The inactive position of the needle drawing assistance apparatus 10 is defined where the first planar member 11 rests substantially parallel and in contact with the second planar member 14. The inactive position allows for enhanced storability and usability to a user. For example, the user may not need to move the needle drawing assistance apparatus 10 into the active position in order to extract liquid components from the storage element but may desire to have the additional stability provided by the first clamp 18 and the second clamp 23 when removing liquid components.

In the illustrated embodiment, the needle drawing assistance apparatus 10 further comprises a pad 31 disposed on a bottom surface of the second planar member 14. The pad 31 is made of a material having a friction coefficient greater than one. When the pad 31 is disposed on the bottom surface of the second planar member 14, greater frictional support is provided to the needle drawing assistance apparatus 10.

Additionally, in the illustrated embodiment, the needle drawing assistance apparatus 10 further comprises at least one light 32. In the illustrated embodiment, two lights 32 are disposed on the top surface of the first planar member 11. The lights 32 are disposed between the first clamp 18 and the second clamp 23, such as to provide enhanced vision to a user when utilizing a needle to draw fluid from a storage element. The lights 32 are in operable connection with an actuator 33. The actuator 33 is configured to be actuatable by a user such that the lights 32 can be freely turned on and off.

In the illustrated embodiment, a charging port 37 extends outwardly from the first planar member 11. The charging port 37 is configured to attach to a charger, such as a DC electronic charger, such that the needle drawing assistance device 10 can be charged via a standard electronic outlet. Furthermore, the charging port 37 is in operable connection with a power source 36. In the illustrated embodiment, the power source 36 is a rechargeable battery. As shown, the rechargeable battery is stowed in a cavity defined in the first planar member 11. The cavity is any suitably dimensioned receptacle for receiving the rechargeable battery, such as a cutout in an underside surface of the first planar member 11.

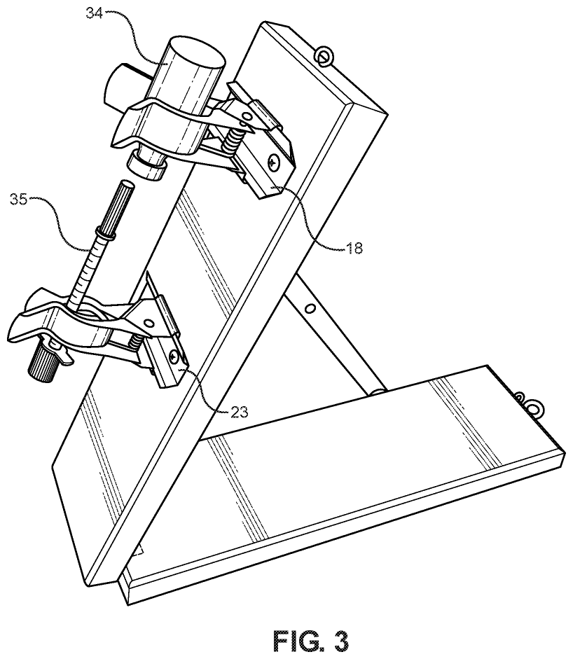

Referring now to FIG. 3, there is shown a perspective view of an embodiment of the needle drawing assistance apparatus in use. In use, a storage element 34 is inserted into the first clamp 18. A needle 35 is inserted into the second clamp 23. The first clamp 18 and the second clamp 23 are oriented such that the needle 35 is in linear alignment with the storage element 34. The first planar member can be elevated, such that the storage element 34 is raised relative to the needle 35 and the needle 35 can extract the contents of the storage element 34 into the reservoir thereof.

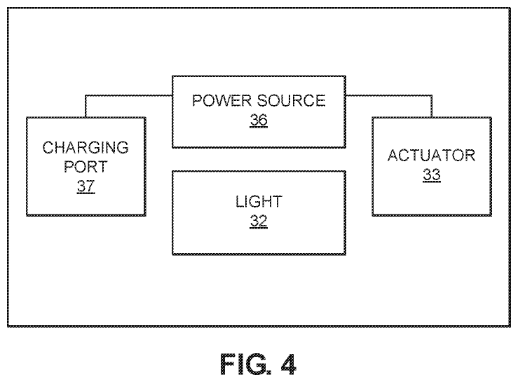

Referring now to FIG. 4, there is shown a block diagram of an embodiment of the needle drawing assistance apparatus. As shown, the light 32 is in operable connection with the actuator 33 and a power source 36. As such, the light 32 is powered by the power source 36 and is actuatable via the actuator 33. In one embodiment, the power source 36 is in operable connection with a charging port 37, such that the power source 36 can be recharged after the amount of energy within the power source 36 is exhausted. The charging port 37 is any means capable of transmitting power from a charging source to the power source 36 of the light 32.

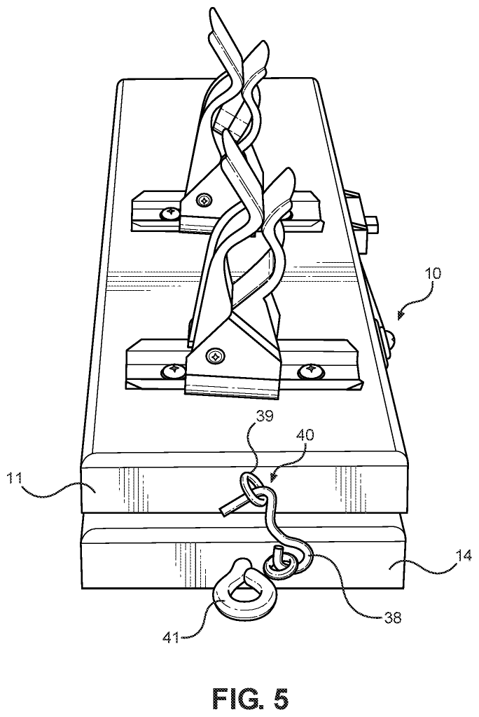

Referring now to FIG. 5, there is shown a rear view of an embodiment of the needle drawing assistance apparatus. In the illustrated embodiment, the needle drawing assistance apparatus 10 further comprises a fastener 40. In the illustrated embodiment, the fastener 40 comprises a hook 38 and a loop 39. The hook 38 is movably mounted upon the distal end of the second planar board 14, such that the hook 38 is configured to be movable into the loop 39. The loop 39 is mounted on the second end of the first planar member 11. When the hook 38 is engaged upon the loop 39, the needle drawing assistance apparatus 10 is secured in an inactive position as shown.

In the illustrated embodiment, a hanger 41 is disposed on the distal end of the second planar member. As such, the needle drawing assistance apparatus 10 may be secured to an upstanding surface via the hanger 41, such as a wall. When placed upon an upstanding surface, the needle may be oriented relative to a storage element in a manner in which gravity will assist in the extraction of material from the storage element.

It is therefore submitted that the instant invention has been shown and described in various embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

We claim:

1) A needle drawing assistance apparatus, comprising:
a first planar member defining a first end and a second end;
a second planar member having a proximal end and a distal end;
the second planar member secured to the first end of the first planar member at the proximal end of the second planar member via a hinge;
the hinge configured to allow the needle drawing assistance apparatus to be movable between an active position and an inactive position;
the inactive position defined where the first planar member rests parallel and in contact with the second planar member;
the active position defined where the first planar member defines an angle greater than zero degrees with the second planar member;
a first clamp disposed on a top surface of the first planar member adjacent to the second end of the first planar member;
a second clamp disposed on the top surface of the first planar member between the first clamp and the first end of the first planar member.

2) The needle drawing assistance apparatus of claim 1, wherein the first clamp and the second clamp are spring biased towards a closed position.

3) The needle drawing assistance apparatus of claim 1, wherein a brace is disposed between the first planar member and the second planar member, the brace configured to secure the needle drawing apparatus in an active position.

4) The needle drawing assistance apparatus of claim 3, wherein the brace comprises a rotatable joint.

5) The needle drawing assistance apparatus of claim 1, wherein a pad is disposed on a bottom surface of the second planar member.

6) The needle drawing assistance apparatus of claim 1, wherein a fastener is disposed on the second end of the first planar member and the distal end of the second planar member, the fastener configured to secure the needle drawing apparatus in a closed position.

7) The needle drawing assistance apparatus of claim 6, wherein the fastener comprises a hook and a loop.

8) The needle drawing assistance apparatus of claim 1, wherein at least one light is disposed on a top surface of the first planar member.

9) The needle drawing assistance apparatus of claim 8, wherein the light is in operable connection with a power source disposed in the first planar member.

10) The needle drawing assistance apparatus of claim 8, wherein the light is in operable connection with an actuator.

11) The needle drawing assistance apparatus of claim 1, further comprising a hanger disposed on the distal end of the second planar member.
